(19) United States
(12) Plant Patent Application Publication
(10) Pub. No.: US 2025/0386749 P1
(43) Pub. Date: Dec. 18, 2025

(54) Echinacea Plant Named 'BullEchipur 118'

(50) ;

(71) Applicant: Hartwig Bull, Gönnebek (DE)

(72) Inventor: Hartwig Bull, Gönnebek (DE)

(21) Appl. No.: 19/235,107

(22) Filed: Jun. 11, 2025

Related U.S. Application Data
(60) Provisional application No. 63/731,837, filed on Jun. 12, 2024.

(51) Int. Cl. A01H 6/14; A01H 5/02

(52) U.S. Cl. CPC A01H 6/1448; A01H 5/02
USPC PLT/428

(57) ABSTRACT

A new and distinct variety of Echinacea plant named ‘BullEchipur 118’ having large pink flowers, good growth habit, enhanced uniformity, good branching, and good propagation rates.

### CROSS REFERENCE TO RELATED APPLICATIONS

This application claims the benefit of priority from U.S. Provisional Patent Application No. 63/731,837, filed Jun. 12, 2024, which is incorporated herein by reference in its entirety.

Botanical classification: Echinacea purpurea.

Varietal denomination: ‘BULLECHIPUR 118’.

### BACKGROUND OF THE INVENTION

The present invention relates to a new and distinct cultivar of Echinacea botanically known as Echinacea purpurea, and hereinafter referred to by the name ‘BullEchipur 118’.

The new Echinacea was discovered by the Inventor in August 2021, in a controlled environment in Gönnebek, Germany, as a part of a planned breeding program. The new Echinacea was selected on the basis of its large pink flowers, good growth habit, enhanced uniformity, good branching, and good propagation rates.

Asexual propagation of the new cultivar by in vitro culture since September 2021, in Gönnebek, Germany, has shown that the unique features of this new Echinacea are stable and reproduced true to type in successive generations.

### SUMMARY OF THE INVENTION

The following traits have been repeatedly observed and are determined to be unique characteristics of ‘BullEchipur 118’. These characteristics in combination distinguish ‘BullEchipur 118’ as a new and distinct cultivar:
1. Compact growths
2. Vibrant pink flowers
3. Lots of large flowers
4. More branching habit
5. Very uniform growth
6. Nice green leaves
7. Fast crop time

In side-by-side comparisons conducted by the Inventor in Gönnebek, Germany, plants of the new Echinacea differed from plants of the female parent, Bul 2020-03, in the following characteristics:
1. More compact
2. More intensive pink flower color
3. Good branching
4. Green small leaves

In side-by-side comparisons conducted by the Inventor in Gönnebek, Germany, plants of the new Echinacea differed from plants of the male parent, Bul 2020-18, in the following characteristics:
1. More compact
2. More intensive pink flower color
3. More branches
4. Lots of flowers
5. Earlier flowering

Plants of the new Echinacea can be compared to the plants of the Echinacea cultivar ‘Pow Wow Wildberry’, believed to be unpatented. In side-by-side comparisons conducted by the Inventor in Gönnebek, Germany, plants of the new Echinacea differed from plants of the cultivar ‘Pow Wow Wildberry’ in the following characteristics:
1. More compact
2. More branching habit
3. More uniform growth
4. More flowers
5. Cleaner leaves
6. Shorter plant

### DESCRIPTION OF THE DRAWINGS

The accompanying colored photographs illustrate the overall appearance of the new Echinacea showing the colors as true as it is reasonably possible to obtain in colored reproductions of this type. Colors in the photographs may differ slightly from the color values cited in the detailed botanical description, which accurately describes the colors of the new Echinacea.

FIG. 1 comprises a top perspective view of a typical plant of ‘BullEchipur 118’ under natural light.

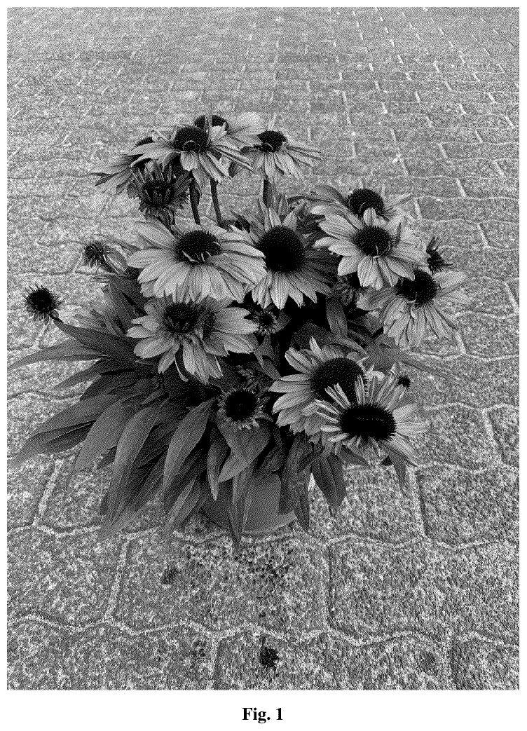

### DESCRIPTION OF THE PLANT

The cultivar Echinacea has not been observed under all possible environmental conditions. The phenotype may vary somewhat with variations in environment such as temperature and light intensity without, however, any variance in genotype.

The aforementioned photographs and following observations and measurements describe plants of the new Echinacea that were grown in an outdoor nursery in Gönnebek, Germany. During the production of the plants, temperatures averaged about 18-20° C. Plants used for the photographs and description were about 22 weeks from planting. Color references are made to The Royal Horticultural Society Colour Chart, 2015 version, except where general terms of ordinary dictionary significance are used.

### PLANT

Botanical classification: Echinacea purpurea.
Parentage: Bul 2020-03 and Bul 2020-18.
Propagation:
   Time to initiate roots.—About 7 days at about 19-20° C.
   Time to develop roots.—About 40-50 days at about 18° C.
   Time to produce a finished flowering plant.—About 12-14 weeks.
   Root description.—Lots of roots, free-branching, nice, healthy-looking root system.
Plant description:
   Growth rate/vigor.—Compact/upright.
   Plant height.—40-50 cm.
   Plant width.—35-40 cm.
   Branching habit.—Lots of branches, upright.
   Lateral branches.—Number per plant: 18-25. Length: 2-33 cm. Diameter: 3-8 cm. Internode length: 1-5 cm. Angle: Upright, outwarding. Strength: Strong. Texture: Pubescent. Color: 146C to 184C.
   Foliage.—Length: 3-21 cm. Width: 1-5 cm. Shape: Lanceolate. Shape of Apex: Acuminate. Shape of Base: Attenuate to Acute. Orientation and aspect: Straight to curved. Texture: Upper surface: Rough. Lower surface: Rough. Young leaves: Upper surface color: 137A. Lower surface color: 137C. Fully expanded leaves: Upper surface color: 137A. Lower surface color: 137C. Venation: Type: Cross venulate to longitudinal. Color: Upper surface: 145A. Lower surface: 145B. Margin: Entire.
Flowers:
   Bud.—Stage of opening: Single type blossom. Shape: Elliptic shaped ray florets and tubular disk floret. Diameter: 1-3.5 cm. Length: 1.2-2.5 cm. Color: When closed: N137C. When open: 187A.
   Natural flowering season.—July-September 13-14 weeks after planting.
   Inflorescence.—Form: Single flower. Disc and ray floret arrangement: Single composite flower. Average number per plant: 20-30.Average number per branch: 1-3.Fragrance: None. Diameter: 4-10 cm. Height: 2-4 cm. Diameter of disc: 1.5-4 cm. Receptacle height: 1 cm. Receptacle diameter: 1.5 cm-4 cm. Ray florets: Number per inflorescence: 16-24.Arrangement: In 2-3 whorls. Length: 1.5-4.5 cm. Width: 0.9-1.7 cm. Shape: Ellipse. Shape of Apex: Obtuse. Shape of Base: Acute. Margin: Entire. Texture: Upper surface: Smooth, glabrous. Lower surface: Rough. Color: When opening: Upper surface: 72C. Lower surface: 73A. At maturity: Upper surface: 72C. Lower surface: 73A. Ground color description: Pink. Disc florets: Number per inflorescence: Numerous, too many to quantify. Arrangement: In center of receptacle. Length: 8-15 mm. Width: 1-2 mm. Shape: Tubular. Shape of Apex: Sharp. Color: Immature: 187B. At maturity: 187B. Phyllaries: Number per inflorescence: 35-50.Arrangement: In 3-4 whorls. Length: 0.5-1.5 cm. Width: 2-3 mm. Shape: Lanceolate. Shape of Apex: Elongate, oval. Shape of Base: Fused. Margin: Entire. Texture: Upper surface: Rough. Lower surface: Rough. Color: Immature: Upper surface: NN137C. Lower surface: NN137C. At maturity: Upper surface: NN137C. Lower surface: NN137C.
Reproductive organs:
   Androecium.—Presence: On disk florets. Number per floret: 1.Filament: Length: 1-2 mm. Color: 187B. Anther: Shape: Oval. Length: 1 mm. Color: 187B. Pollen: Color: 15B. Amount: Too much to quantify.
   Gynoecium.—Presence: In disc and ray florets. Pistil length: 1-2 mm. Stigma: Shape: Two-parted. Color: 187B. Style: Length: 1-2 mm. Color: 187B.
   Seeds/fruit.—Length: 1-2 mm. Color:
Disease/pest resistance:
   Disease resistance.—No additional disease resistance has been observed beyond the typical resistance in Echinacea plants.
   Pest resistance.—No additional pest resistance has been observed beyond the typical resistance in Echinacea plants.
Weather tolerance:
   Temperature tolerance.—No additional temperature resistance has been observed beyond the typical resistance in Echinacea plants.
   Drought tolerance.—No additional drought resistance has been observed beyond the typical resistance in Echinacea plants.

I claim:

1. A new and distinct cultivar of Echinacea plant named ‘BullEchipur 118’, as illustrated and described.
